(19) United States
(12) Patent Application Publication
(10) Pub. No.: US 2005/0220899 A1
(43) Pub. Date: Oct. 6, 2005

(54) Use of cisplatin in combination with folic acid for increasing cisplatin efficacy

(76) Inventor: Clet Niyikiza, Indianapolis, IN (US)

Correspondence Address: ELI LILLY AND COMPANY;PATENT DIVISION, P.O. BOX 6288, INDIANAPOLIS, IN, 46206-6288, US

(21) Appl. No.: 10/513,230

(22) PCT Filed: May 1, 2003
(86) PCT No.: PCT/IB03/01715

§ 371 Date: Nov. 1, 2004

Related U.S. Application Data
(60) Provisional application No. 60/378,923, filed on May 7, 2002.

(51) Int. Cl. A61K 33/24; A61K 31/525

(52) U.S. Cl. 424/649; 514/251

(57) ABSTRACT

The present invention thus provides a method for improving the therapeutic utility of cisplatin by administering to the host undergoing treatment with a cisplatin a low dose of folic acid.

Cisplatin (cis-diamminedichloroplatinum, cis-Pt(NH₃)₂Cl₁₂, molecular weight 300.05) has been used as a chemotherapeutic agent for many years since the discovery of its antitumor activity by B. Rosenberg, et al. (Nature, 1965, 205, 698) Cisplatin is referred to commercially as Platinol® and is used to treat such cancers as testicular, ovarian, and bladder. (Physician's Desk Reference, PDR) Although cisplatin has been used for many years to treat such cancers, more effective treatments are continually being sought. Many attempts have been made to modify the cisplatin molecule in order to reduce toxicity and increase efficacy and a few attempts have been made to modify the composition of cisplatin dosage form to reduce toxicity and improve its efficacy.

For example, the combination of high dosages of folic acid with cisplatin has shown to potentially reduce the toxicity of cisplatin. (See, e.g. U.S. Pat. No. 6,297,245, J. Shaw.) Shaw teaches that a pharmaceutical composition comprising between 1:0.05 and 1:1 mole ratio of cisplatin to folic acid reduces the toxicity of cisplatin. Additionally, Kurbacher C M, et al. suggest that the combination of ascorbic acid and cisplatin may enhance the efficacy of cisplatin. (Kurbacher C M, Wagner U, Kolster B, et al. Ascorbic acid (vitamin C) improves the antineoplastic activity of doxorubicin, cisplatin, and paclitaxel in human breast carcinoma cells in vitro. Cancer Lett 1996;103:183-189.)

However, even with these advancements, an increase in the efficacy of cisplatin would be a significant development breakthrough. By increasing efficacy, less drug substance would need to be administered to achieve the same therapeutic response, or the same dose of the drug could be administered with an enhanced effect. Therefore, the ability to increase efficacy of cisplatin would represent an important advance in the use of this agent.

The prior art neither suggests nor discloses the use of low dosages of folic acid to enhance the therapeutic efficacy of cisplatin. Surprisingly and unexpectedly, we have now discovered just that: administering cisplatin in combination with a low dose of folic acid can enhance the therapeutic efficacy of cisplatin. The present invention thus provides a method for improving the therapeutic utility of cisplatin by administering to the host undergoing treatment with a cisplatin a low dose of folic acid.

The present invention relates to a method of administering cisplatin in combination with folic acid, wherein the mole ratio of cisplatin to folic acid is about 1:0.002 to about 1:0.015.

Furthermore, the present invention relates to a method of enhancing the therapeutic efficacy of cisplatin comprising administering to said mammals cisplatin in combination with folic acid, wherein the mole ratio of cisplatin to folic acid is about 1:0.002 to about 1:0.015.

Furthermore, the present invention relates to a use of folic acid in the preparation of a medicament useful in increasing the therapeutic efficacy of cisplatin in a human, and the medicament is for administration in combination with cisplatin, and in a mole ratio of cisplatin to folic acid of about 1:0.002 to about 1:0.015.

The current invention concerns the discovery that therapeutic efficacy can be enhanced by administering cisplatin in combination with folic acid, wherein the mole ratio of cisplatin to folic acid is about 1:0.002 to about 1:0.015, preferably about 1:0.003 to about 1:0.010, and most preferably about 1:0.005 to about 1:0.008.

As used herein, the term “therapeutic efficacy” refers to cisplatin's ability to control a subject's disease state or produces a beneficial result in such disease state.

As used herein, the term “in combination with” refers to the administration of cisplatin with folic acid; in any order such that sufficient levels of folic acid are present to enhance the therapeutic efficacy of cisplatin. Cisplatin may be administered to the mammal first, followed by treatment with folic acid. Alternatively, the mammal may be administered cisplatin simultaneously with folic acid. Preferably, the mammal is pretreated with the folic acid and then administered cisplatin.

In general, the term “pharmaceutical” when used as an adjective means substantially non-toxic to living organisms.

Folic acid is commercially available from such sources as Spectrum Quality Products, Inc. and Sigma Chemical Company. Preferably, folic acid is administered to the subject orally. It will be understood that the amount of the folic acid actually administered will be determined by a physician, in the light of the relevant circumstances, including the condition to be treated, the chosen route of administration, the age, weight, and response of the individual patient, and the severity of the patient's symptoms. Therefore the dosage ranges exemplified are not intended to limit the scope of the invention in any way. In some instances dosage levels below the lower limit of the aforesaid range may be more than adequate, while in other cases still larger doses may be employed without causing any harmful side effect.

Cisplatin is commercially available from many sources such as Sigma Chemical Company and Alfa Aesar. Preferably, cisplatin is administered as a parenteral. However, it will be understood that the amount of the cisplatin actually administered will be determined by a physician, in the light of the relevant circumstances, including the condition to be treated, the chosen route of administration, the age, weight, and response of the individual patient, and the severity of the patient's symptoms. Therefore the dosage ranges exemplified are not intended to limit the scope of the invention in any way. In some instances dosage levels below the lower limit of the aforesaid range may be more than adequate, while in other cases still larger doses may be employed without causing any harmful side effect.

The key to the present invention is to maintain the mole ratio of cisplatin to folic acid in the range of about 1:0.002 to about 1:0.015.

Current studies have shown that 59 human patients exhibiting mesothelioma treated with 75-120 mg/m² of cisplatin without folic acid exhibited a median survival time of 7.2 months and a response rate of 8.5%. However, in a study of 163 human patients exhibiting mesothelioma treated with 75-120 mg/² of cisplatin in combination with 350-1000 μg of folic acid, the group exhibited an enhanced median survival time of 10.0 months and a response rate of more than double the unsupplemented trial group; 19.6%.

1. A method of administering cisplatin in combination with folic acid, wherein the mole ratio of cisplatin to folic acid is about 1:0.002 to about 1:0.015.

2. The method of claim 1 wherein the mole ratio of cisplatin to folic acid is about 1:0.003 to about 1:0.010.

3. The method of claim 1 wherein the mole ratio of cisplatin to folic acid is about 1:0.005 to about 1:0.008.

4. A method of enhancing the therapeutic efficacy of cisplatin comprising administering to a mammal cisplatin in combination with folic acid, wherein the mole ratio of cisplatin to folic acid is about 1:0.002 to about 1:0.015.

5. The method of claim 4 wherein the mole ratio of cisplatin to folic acid is about 1:0.003 to about 1:0.010.

6. The method of claim 5 wherein the mole ratio of cisplatin to folic acid is about 1:0.005 to about 1:0.008.

7. The method of any one of claim 6 wherein the mammal is human.

8. The use of folic acid in the preparation of a medicament useful in increasing therapeutic efficacy in a human: of cisplatin, and the medicament is for administration in combination with cisplatin, and in a mole ratio of cisplatin to folic acid of about 1:0.002 to about 1:0.015.

9. The use of claim 8 wherein the mole ratio of cisplatin to folic acid is about 1:0.003 to about 1:0.010.

10. The use of claim 9 wherein the mole ratio of cisplatin to folic acid is about 1:0.005 to about 1:0.008.
